(19) United States
(12) Patent Application Publication
(10) Pub. No.: US 2005/0064012 A1
(43) Pub. Date: Mar. 24, 2005

(54) Process for causing myopic shift in vision

(75) Inventors: Ronald L. Gross, Houston, TX (US); Stephen C. Pflugfelder, Houston, TX (US)

Correspondence Address: BAKER BOTTS LLP;C/O INTELLECTUAL PROPERTY DEPARTMENT, THE WARNER, SUITE 1300, 1299 PENNSYLVANIA AVE, NW, WASHINGTON, DC, 20004-2400, US

(73) Assignee: Baylor College of Medicine, Houston, TX (US)

(21) Appl. No.: 10/928,738

(22) Filed: Aug. 30, 2004

Related U.S. Application Data
(63) Continuation-in-part of application No. 10/174,983, filed on Jun. 20, 2002.
(60) Provisional application No. 60/498,325, filed on Aug. 28, 2003.
(60) Provisional application No. 60/305,618, filed on Jul. 17, 2001.

(51) Int. Cl. A61B 18/18; A61F 2/00

(52) U.S. Cl. 424/428

(57) ABSTRACT

A process for causing a myopic shift in the vision of a patient's eye, in which the patient's eye includes a cornea includes the step of instilling a bio-compatible material to the cornea of the eye, in which the bio-compatible material includes bio-compatible molecules and the cornea has a first composite refractive index. In addition, the bio-compatible molecules alter the first composite refractive index to a second composite refractive index.

The present application claims priority from U.S. Provisional Patent Application No. 60/498,325, filed Aug. 28, 2003, and is a continuation-in-part of U.S. patent application Ser. No. 10/174,983, filed Jun. 20, 2002, which claims priority from U.S. Provisional Patent Application No. 60/305,618, filed Jul. 17, 2001. The disclosure of each of these earlier applications is incorporated herein by reference in its entirety.

### BACKGROUND OF THE INVENTION

1. Field of the Invention

The invention relates generally to a process for causing a myopic shift in the vision of a patient's eye. Specifically, the invention is directed to a process for causing a myopic shift in the vision of a patient's eye by instilling a bio-compatible molecule to the cornea of the patient's eye.

2. Description of Related Art

Ametropia is a generic term which refers to any condition of imperfect refraction in the eyes, such as nearsightedness, farsightedness (e.g., presbyopia), or any astigmatism. In particular, presbyopia is the reduced ability to focus on near objects caused by loss of elasticity in the corneal lens with increased age. It is known that refraction of the eyes may be manipulated by altering the curvature of the cornea of the eye. For example, contact lenses may be effective in correcting the effects of an ametropia by manipulating the effective curvature of the cornea of the eye. However, a disadvantage of contact lenses, whether hard or soft contact lenses, is that wearers may experience a degree of discomfort when the lenses are worn. In addition, contact lenses may be difficult to insert or remove from the eye, and also may be easily lost or misplaced. Eyeglasses also may be effective in correcting the effects of an ametropia by manipulating the effective curvature of the eye. However, glasses may be easily lost, misplaced or broken. Glasses also may be cumbersome to wear, and, for some patients, they are cosmetically unacceptable. In addition, if a wearer of the contact lenses or glasses forgets to wear the contact lenses or forgets to bring the glasses with him or her when he or she travels, he or she may suffer from the effects of an ametropia during the time that he or she is not wearing or able to wear the contact lenses or glasses.

Various surgical procedures also may be available to correct an ametropia by manipulating the curvature of the corneal lens of the eye. However, even the simplest surgical procedure presents some risk of permanent damage to a patient undergoing the surgical procedure due to error or infection. Moreover, such surgical procedures may be expensive, and they frequently are irreversible. In addition, the patient is likely to undergo post-surgical discomfort and also may continue to experience discomfort, such as irritation of the eye, well after the completion of the surgical procedure.

Various implant lenses also may be available to correct an ametropia by replacing the crystalline lens of the eye. However, such lens implants involve surgical implantation, which again presents some risks of infection and permanent damage to the eye.

### SUMMARY OF THE INVENTION

Therefore, a need has arisen for a process that overcomes these and other shortcomings of the related art. A technical advantage of the present invention is that patients may achieve a myopic shift in their vision without having to undergo surgical procedures and, in particular, without having to receive lens implants. Myopia may be defined as a spherical equivalent of less than −0.5 diopters (D). Hyperopia may be defined as a spherical equivalent of greater than +0.5 D. When discussing the direction of shift in spherical equivalents, a myopic shift is a shift in the negative direction. Another technical advantage is that patients may improve their vision without using contact lenses or glasses. Yet another technical advantage of the present invention is that patients temporarily may achieve a myopic shift in their vision when they forget or lose their contact lenses or glasses, or if they do not wish to wear their contact lenses or glasses for a predetermined length of time.

In an embodiment of the present invention, a process for causing a myopic shift in the vision of a patient's eye, in which the patient's eye comprises a cornea, is described. The process comprises the step of instilling a bio-compatible material to the cornea of the eye, in which the cornea has a first composite refractive index, and the bio-compatible material comprises a plurality of bio-compatible molecules. In addition, the bio-compatible molecules alter the first composite refractive index to a second composite refractive index.

In another embodiment of the present invention, a process for causing a myopic shift in the vision of a patient's eye, in which the patient's eye comprises a cornea, is described. The process includes the step of instilling a bio-compatible material to the cornea of the eye, in which the cornea has a first composite refractive index, and the bio-compatible material comprises a plurality of bio-compatible molecules. In addition, the bio-compatible molecules alter the first composite refractive index to a second composite refractive index for a predetermined length of time.

Other objects, features, and advantages will be apparent to persons of ordinary skill in the art in view of the following detailed description of the invention and the accompanying drawings.

### BRIEF DESCRIPTION OF THE DRAWINGS

For a more complete understanding of the present invention, the needs satisfied thereby, and the features and advantages thereof, reference now is made to the following descriptions taken in connection with accompanying drawings.

FIG. 1 is schematic of a cornea of a patient's eye according to an embodiment of the present invention.

FIG. 2 is a schematic of a process according to an embodiment of the present invention.

FIG. 3 is a chart depicting the results of a first experiment conducted according to the process of FIG. 2.

FIG. 4 is a graph depicting the results of a second experiment measuring the spherical equivalent in diopters for test subjects right eyes whose were treated with a measured amount of a liquid containing carboxymethylcellulose (CMC).

FIG. 5 is a graph depicting the results of the second experiment measuring the spherical equivalent in diopters for test subjects left eyes whose were treated with a measured amount of a gel containing sodium hyaluronate (SH).

FIG. 6 is a chart depicting the average myopic shift from baseline for each set of eyes in the second experiment.

FIG. 7 is a graph depicting the results of a third experiment measuring the average corneal power in diopters for test subjects whose right eyes were treated with a measured amount of a liquid containing CMC and whose left eyes were treated with a measured amount of a gel containing SH.

### DETAILED DESCRIPTION OF PREFERRED EMBODIMENTS

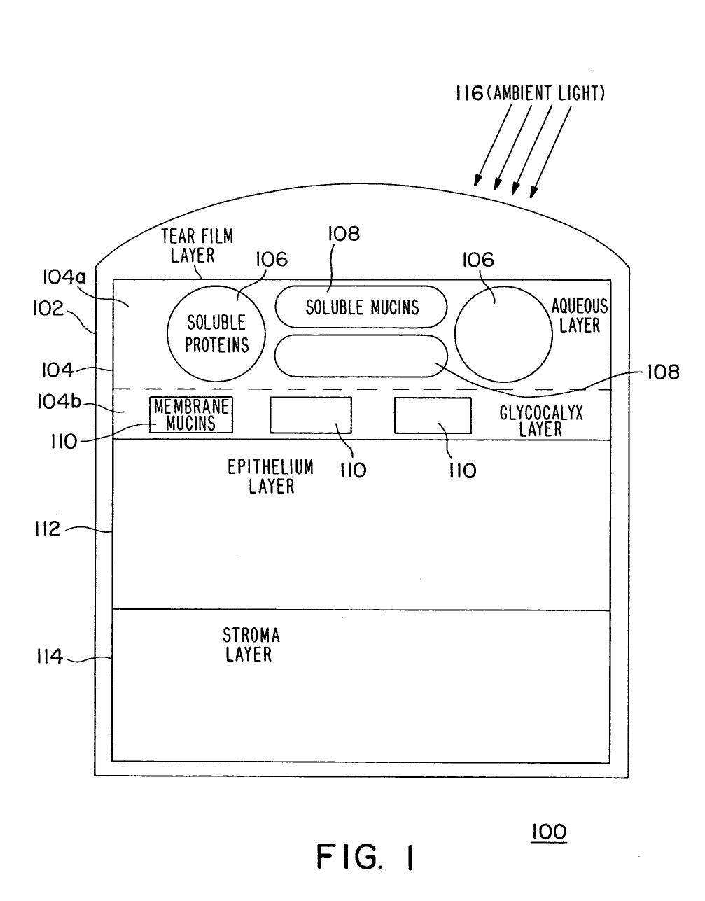

Preferred embodiments of the present invention and their advantages may be understood by referring to FIGS. 1-7, like numerals being used for like corresponding parts in the various drawings.

Referring to FIG. 1, a schematic for a patient's eye 100 is described. A patient may have eye 100, which may comprise a cornea 102 and an inferior conjunctive sac or lower eye-lid 103. Cornea 102 may comprise a tear film layer 104, an epithelium layer 112, and a stroma layer 114. While not wishing to be bound by a theory, it is believed that tear film layer 104 may comprise an aqueous or tear mucin layer 104a and a glycocalyx layer or coating 104b. Moreover, aqueous layer 104a may comprise a plurality of soluble proteins 106 and a plurality of soluble mucins 108, and glycocalyx layer 104b may comprise a plurality of membrane mucins 110. For example, See, e.g., Stephen C. Pflugfelder et al, Detection of Sialomucin Complex (MUC4) in Human Ocular Surface Epithelium and Tear Fluid, Investigative Ophthalmology & Visual Science, May 2000, Volume 41, No. 6, at 1316, the disclosure of which is incorporated herein by reference in its entirety. In this embodiment, ambient light 116 first may enter cornea 102 through tear film layer 104.

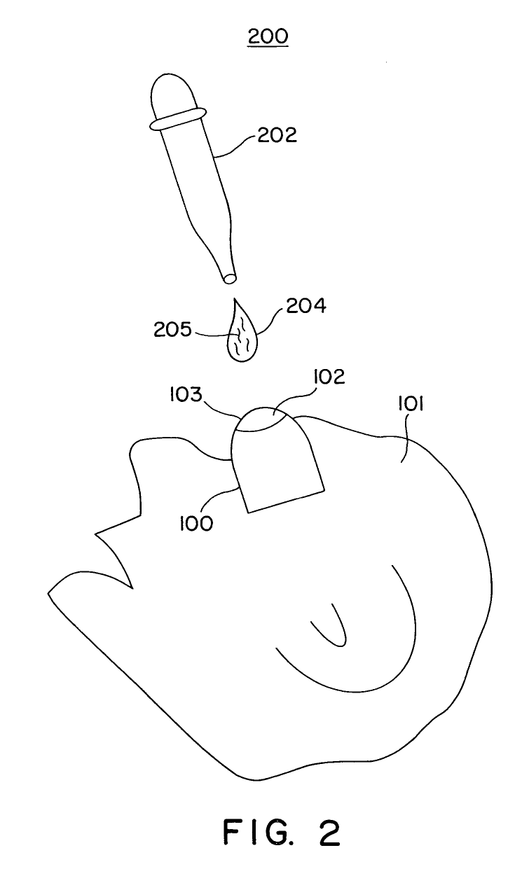

Referring to FIG. 2, a schematic of a process 200 for causing a myopic shift in the vision of eye 100 of a patient 101 according to an embodiment of the present invention is described. The process comprises the step of instilling a bio-compatible material 204 to cornea 102 of eye 100. It will be understood by those of ordinary skill in the art that the term “instill” may include any known means for contacting bio-compatible material 204 with cornea 102 of eye 100, such as via an eye dropper 202, an aerosol sprayer (not shown), or the like. Moreover, the term “bio-compatible” may include any material which may be suitable for instillation in eye 100. Bio-compatible material 204 may comprise a plurality of bio-compatible molecules 205 and at least a portion of bio-compatible material 204 may be instilled on inferior conjunctive sac 103 by eye dropper 202, such that at least a portion of bio-compatible molecules 205 may permeate tear film 104, as depicted in FIG. 1. In an embodiment, bio-compatible material 204 may be a hydrophilic polyanionic diaccharide, such as a sodium hyaluronate gel. Nevertheless, it will be understood by those of ordinary skill in the art that bio-compatible material 204 may be any bio-compatible material which may alter the composite refractive index of cornea 102, eg., a bio-compatible material comprising bio-compatible molecules which may form a chemical attraction with molecules in tear film 104, such as glycocalyx or epithelial cells. For example, the sodium hyaluronate gel may be about 1% sodium hyaluronate gel, and an amount in a range of about 0.05 ml to about 0.1 ml of the 1% sodium hyaluronate gel may be instilled on inferior conjunctive sac 103. In an embodiment of the invention, the 1% sodium hyaluronate gel may be Healon® gel, which is available from Pharmacia of Peapack, New Jersey, USA. Moreover, the strength of the chemical attraction between the molecules and the cells may alter the duration and the degree of the myopic shift.

In addition, cornea 102 may have a first composite refractive index and a first optical power. The first composite refractive index of cornea 102 may be a summation of the refractive indices of the various layers of cornea 102. For example, the refractive index of tear film layer 104 may be between about 1.3357 and about 1.3374, and the refractive index of epithelium layer 112 and stroma layer 114 may be about 1.3760. In this embodiment, the first composite refractive index of cornea 102 may be about 1.3760. Moreover, the first optical power of cornea 102 may be related to the first composite index of refraction of cornea 102. For example, the first optical power (i e., corneal power) of cornea 102 may be estimated using the formula:
Optical Power=(α−β)/γ
in which α is the first composite refractive index of cornea 102, β is the refractive index of air, i.e., one (1), and γ is the radius of curvature of the anterior corneal surface (not shown). In this embodiment, when the radius of curvature of the anterior corneal surface is about 7.8 mm, the first optical power may be estimated at about 48.2 D, i.e., (1.3760−1)/0.0078=48.2 D. However, although the estimated first optical power may be estimated at about 48.2 D, the actual or effective first optical power only may be about 43 diopters. The actual first optical power may be less than the estimated first optical power because there may be about a negative six (−6) D lens effect between the posterior cornea (not shown) and the aqueous humor (not shown).

When a measured amount of bio-compatible material 204 comprising the plurality of bio-compatible molecules 205 is instilled in eye 100, bio-compatible molecules 205 may alter a first refractive index of tear film 104 to a second refractive index. For example, while not wishing to be bound by a theory, it is believed that tear film 104 may comprise tear mucin layer 104a and glycocalyx layer 104b. Moreover, tear mucin layer 104a may comprise a plurality of molecules, such as proteins 106 and soluble mucins 108, which may bind bio-compatible molecules 205 to tear film 104. Similarly, glycocalyx layer 104b may comprise membrane mucins 110, which also may bind bio-compatible molecules 205 to tear film 104. Moreover, bio-compatible molecules 205 may have a refractive index which is greater than or less than the refractive index of tear film 104. When bio-compatible molecules 205 bind to tear film 104, because the refractive index of bio-compatible molecules 205 are different than the refractive index of tear film 104, the refractive index of tear film 104 may be altered.

Moreover, altering the refractive index of tear film 104 also may alter the first composite refractive index of cornea 102 to a second composite refractive index. Because the optical power of cornea 102 may be related to the composite refractive index of cornea 102, e.g., the estimated optical power may equal a difference between a composite refractive index of cornea 102 and the refractive index of air divided by the radius of curvature of the anterior corneal surface (not shown), altering the first composite refractive index of cornea 102 to a second composite refractive index also may alter the first optical power of cornea 102 to a second optical power.

For example, the refractive index of bio-compatible molecules 205 may be greater than the refractive index of tear film 104. When bio-compatible molecules 205 bind to tear film 104, because the refractive index of bio-compatible molecules 205 is greater than the refractive index of tear film 104, the refractive index of tear film 104 may be increased. Moreover, increasing the refractive index of tear film 104 also may increase the first composite refractive index of cornea 102 to a second composite refractive index. Because the optical power of cornea 102 is related to the composite refractive index of cornea 102, increasing the first composite refractive index of cornea 102 to a second composite refractive index also may increase the first optical power of cornea 102 to a second optical power. Alternatively, the refractive index of bio-compatible molecules 205 may be less than the refractive index of tear film 104, which may decrease the first composite refractive index of cornea 102 to a second composite refractive index and the first optical power of cornea 102 to a second optical power.

In addition, altering the first composite refractive index of cornea 102 to a second composite refractive index, may increase an ability of eye 100 to focus, which may improve the vision of eye 100. While not wishing to be bound by a theory, it is believed when patient 101 suffers from an ametropia of eye 100, ambient light 116 may not focus on a retina (not shown) of eye 100. For example, if patient 101 suffers from presbyopia or farsightedness, ambient light 116 may focus behind the retina of eye 100. If patient 101 suffers from nearsightedness, ambient light 116 may focus in front of the retina of eye 100. Specifically, ambient light 116 may include portions of light having varying wavelengths, such that the individual portions of ambient light 116 may enter eye 100 at different locations. Because cornea 102 may have an index of refraction which is different than the index of refraction of air, when ambient light 116 enters cornea 102, ambient light 116 may bend. Moreover, the individual portions of ambient light 116 may bend, such that the individual portions of ambient light 116 may converge and intersect at a single focal point. When the natural focal point of patient 101 is a location other than the retina of eye 100, the vision of patient 101 may be imperfect or at least slightly blurred. Altering the first composite refractive index of cornea 102 to the second composite refractive index may alter the focal point of eye 100, such that the focal point may be positioned on the retina of eye 100, which may improve the vision of patient 101.

If patient 101 suffers from presbyopia, farsightedness, or another ametropia in which the focal point of eye 100 is located behind the retina of eye 100, increasing the first composite refractive index of cornea 102 to a second composite refractive index may increase an ability of eye 100 to focus, which may cause a myopic shift in the vision of eye 100. Moreover, it will be understood by one of ordinary skill in the art that the amount by which increasing the first composite index of cornea 102 to the second composite index may increase the ability of eye 100 to focus, may depend on the particular ametropia from which patient 101 suffers.

The measured amount of bio-compatible material 204 containing bio-compatible molecules 205 instilled in eye 100 may vary depending on the particular ametropia from which patient 101 suffers and the severity of the ametropia. For example, a patient suffering from an ametropia which is more sever than an ametropia suffered by another patient, may require a greater amount of bio-compatible material 204 be instilled in eye 100 in order to raise the first composite refractive index of cornea 102 to a second composite index sufficient to position the focal point on the retina of eye 100, such that the ability of the patient to focus may increase to a desired level. For example, in one embodiment, patient 101 may suffer from presbyopia, such that the focal point is positioned behind the retina of eye 100, and cornea 102 may have a first composite index of refraction of about 1.3760. In this embodiment, raising the first composite refractive index of cornea 102 to a second composite refractive index of between about 1.3850 and about 1.4000 may be sufficient to position the focal point on the retina of eye 100. However, it will understood by one of ordinary skill in the art that the measured about of bio-compatible material 204 which may be used to position the focal point on the retina of eye 100 may vary from individual to individual, but may be determined by individual testing and examination.

In another embodiment of the present invention, bio-compatible molecules 205 may alter the first composite refractive index of cornea 102 to the second composite refractive index for a predetermined length of time. Because the optical power of cornea 102 may be related to the composite refractive index of cornea 102, altering the first composite refractive index of cornea 102 to a second composite refractive index for a predetermined length of time also may alter the first optical power of cornea 102 to a second optical power for the predetermined length of time.

While not wishing to be bound by a theory, it is believed that the predetermined length of time may depend on such factors as a temperature and a humidity of an area where patient 101 is located when is instilling bio-compatible material 204 into eye 100. For example, if the temperature or the humidity is greater at a first location than at a second location, bio-compatible material 204 may evaporate or may be excreted from the eye in the form of tears more rapidly at the first location than at the second location. The predetermined length of time also may depend on such factors as the amount bio-compatible material 204 instilled in eye 100 and the actions of patient 101, e.g., physical activity, a rubbing or a blinking of eye 100, or a tearing of eye 100 may decrease the predetermined length of time. For example, the predetermined length of time may be less than or at least equal to about ten (10) minutes or between about ten (10) minutes and one (1) hour. In an embodiment, bio-compatible molecules 205 may increase the first composite refractive index of cornea 102 to the second composite refractive index and also may increase the first optical power of cornea 102 to the second optical power for the predetermined length of time. When patient 101 suffers from presbyopia or farsightedness, increasing the first composite refractive index of cornea 102 to a second composite refractive index may increase an ability of eye 100 to focus for the predetermined length of time, which may cause a myopic shift in the vision of eye 100 for the predetermined length of time.

### EXAMPLES

Embodiments of the present invention will be further clarified by consideration of the following experiments conducted in accord with the above-described embodiments, which is intended to be purely exemplary of the use of the invention.

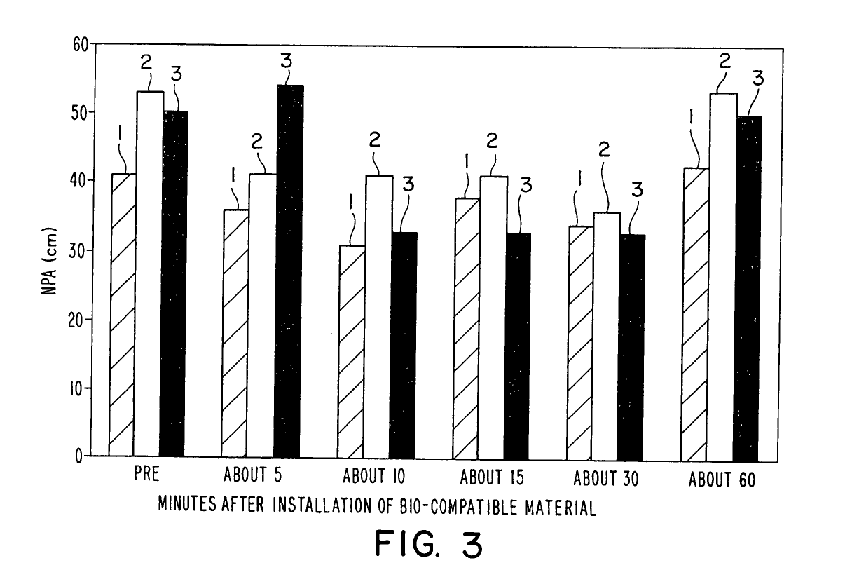

Referring to FIG. 3, a first experiment for treating presbyopia according to the above described embodiments was conducted. This first, conducted experiment involved three subject individuals, a first patient 1, a second patient 2, and a third patient 3, respectively. Initially, the near point of accommodation (NPA), i.e., the point at which a target appears blurred to the individual, was calculated for each of the patients by advancing a fixation target towards the patient and asking them to report when the target appeared blurred. The NPA then was recorded in centimeters and the NPA was recalculated using the same process in order to confirm the accuracy of the calculation. Bio-compatible material 204, which was Healon® gel, then was instilled in conjunctival sac 103 using eye dropper 202 for each of the patients, and the patients were instructed to blink frequently for one (1) minute to facilitate permeation of bio-compatible molecule 204 to tear film 104. NPA measurements then were calculated at about five (5) minutes, about ten (10) minutes, about thirty (30) minutes, and about sixty (60) minutes after the instillation of bio-compatible material 204. As the NPA of a particular patient decreases, the optical power of eye 100 will increase.

As shown in FIG. 3, after about five (5) minutes, patient 1 and patient 2 each experienced a decrease in their NPA, while patient 3 experienced a slight increase in NPA. After about ten (10) minutes, patient 1 experienced a further decrease in NPA, patient 2 experienced substantially no change in NPA between about five (5) and about ten (10) minutes, and patient 3 experienced a decrease in NPA. After about fifteen (15) minutes, patient 1 experienced a slight increase in NPA between about ten (10) and about fifteen (15) minutes and patient 2 and patient 3 experienced substantially no change in NPA between about ten (10) and about fifteen (15) minutes. After about thirty (30) minutes, patient 1 and patient 2 experienced a further decrease in NPA between about fifteen (15) minutes and about thirty (30) minutes, while patient 3 experienced substantially no change between about fifteen (15) minutes and about thirty (30) minutes. Finally, after about sixty (60) minutes, the NPA for each of the patients returned to about the same distance that was calculated for each of the respective patients prior to the instillation of bio-compatible material 204. In this experiment, patient 1 experienced maximum decrease in NPA relative to the NPA calculated prior to instillation of bio-compatible molecule 204 at about ten (10) minutes and patient 2 and patient 3 each experienced their maximum decrease in NPA at about thirty (30) minutes.

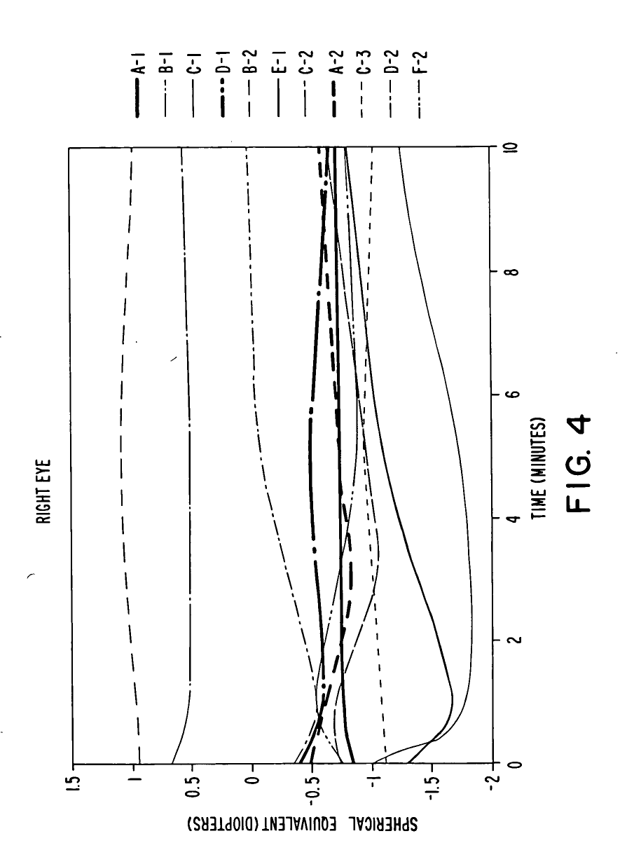

Referring to FIGS. 4-6, a second experiment was conducted to measure the spherical equivalent in diopters for test subjects whose right eyes were treated with a measured amount of a liquid containing CMC and whose left eyes were treated with a measured amount of a gel containing SH, in accordance with embodiments described above. More specifically, the purpose of this second experiment was to evaluate the effect of artificial tears containing CMC and of a gel containing SH on the refractive status, i.e., a spherical equivalent in diopters, of the treated eyes. Measurements were performed using an objective wavefront sensor.

This second, conducted experiment involved six (6) test subjects: a first patient A, a second patient B, a third patient C, a fourth patient D, a fifth patient E, and a sixth patient F, respectively. Initially, a baseline refractive status for each of the test subjects was measured using a Hartmann-Shack wavefront sensor or aberrometer. Such aberrometers are available from several sources, but the aberrometer used in this experiment was the WaveScan™ aberrometer available from VISX Inc. of Santa Clara, Calif., USA. One drop (about 0.05 ml) of Refresh Tears® artificial tear drops, which are available from Allergen, Inc. of Irvine, Calif., USA, and contains about 0.5% CMC, was placed in the right eye of each test subject at time zero; and one drop (about 0.1 ml) of Healon® gel, which contains about 1% SH, was placed in the left eye of each test subject at time zero. The refractive status of each eye was measured at designated time periods of one (1) minute and of three (3), five (5), and ten (10) minutes. The procedure was performed on one (1) test subject once, four (2) test subjects twice, and one (1) test subject three times for a total of twelve (12) procedures. A statistical analysis of the results of these procedures was performed using the paired t-test. The p-level of a t-test represents the probability of error involved in accepting the research hypothesis about the existence of a difference. This is the probability of error associated with rejecting the hypothesis of no difference between the two categories of observations corresponding to the two sets of eyes in the population when, in fact, the hypothesis is true.

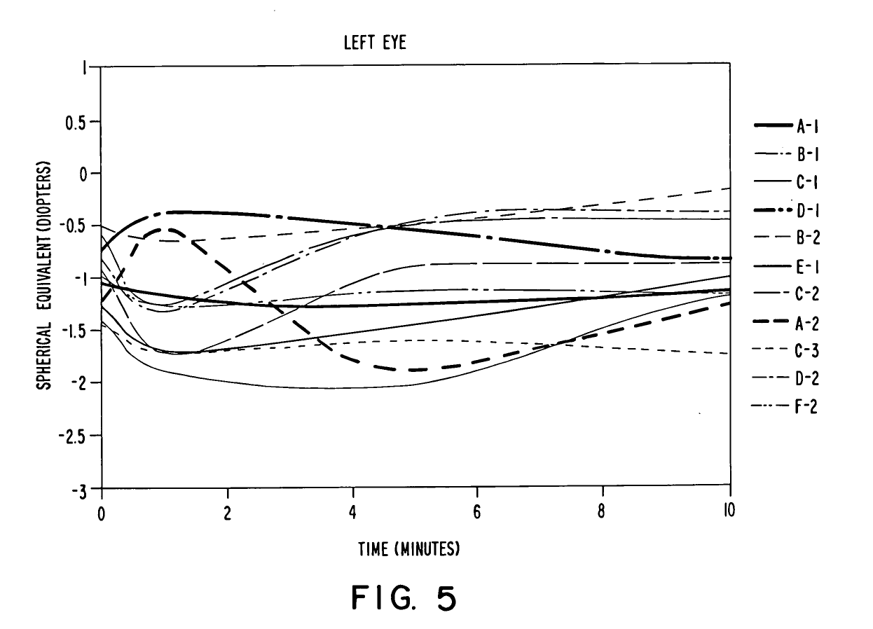

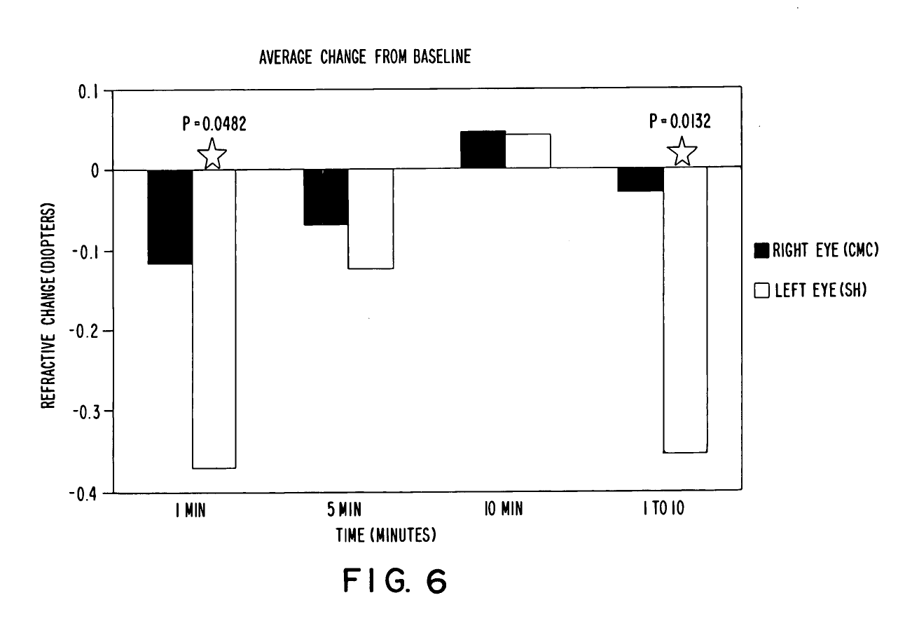

Referring again to FIGS. 4 and 5, the application of a liquid containing CMC and of a gel containing SH to the test subjects' eyes resulted in a detectable myopic shift in the test subjects' vision over the test period. Referring to FIG. 6, however, the myopic shift detected from the application of a gel containing SH was greater and statistically significant when compared to the baseline refractive status for the test subjects. At the one (1) minute period, the p-level for the left eyes of the test subject is 0.0482 indicating that the probability that the results are random is less than 5%. Similarly, over the ten (10) minute test interval, the p-level for the left eyes of the test subject is 0.0132 indicating that the probability that the results are random is less than 2%.

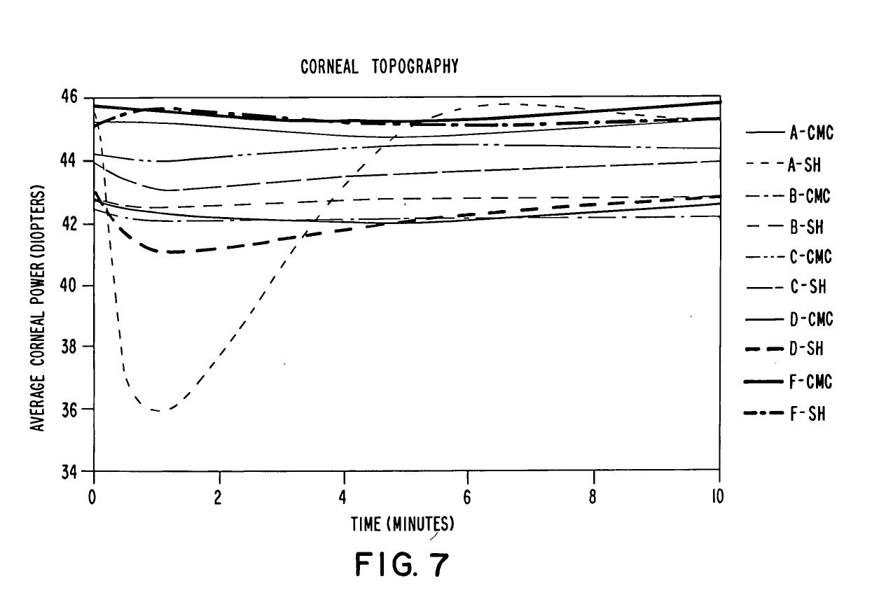

Referring to FIG. 7, a third experiment was conducted to evaluate the effect on the corneal curvature of test subjects whose right eyes were treated with a measured amount of a liquid containing CMC and whose left eyes were treated with a measured amount of a gel containing SH, in accordance with the above described embodiments. More specifically, the purpose of this third experiment was to evaluate the effect of artificial tear drops containing CMC and of a gel containing SH on the corneal power measured in diopters of the treated eyes. Measurements were performed using a computerized videokeratoscopy.

This third, conducted experiment involved five (5) test subjects: first patient A, second patient B, third patient C, fourth patient D, and sixth patient F, respectively. Initially, a baseline corneal power for each of the test subjects was measured using a corneal topographer. Such topographers are available from several sources, but the topographer used in this experiment was the Tomey TMS-2N advanced corneal-topographer available from TOMEY GmbH of Erlangen, Germany. As with the second experiment, one drop (about 0.05 ml) of Refresh Tears® artificial tear drops was placed in the right eye of each test subject at time zero; and one drop (about 0.1 ml) of Healon® gel was placed in the left eye of each test subject at time zero. The corneal power of each eye was measured at designated time periods of one (1) minute and of five (5) and ten (10) minutes. The procedure was performed once on each of the five (5) test subjects. A statistical analysis of the results of these procedures again was performed using the paired t-test. While a consistent myopic shift was not detected in the third experiment, this experiment employed only a limited number of test subjects. While not wishing to be bound by a theory, based on these experimental test results, it appears that the myopic shift is due in larger part to the change in refractive status due to an increase in the refractive index of the tear film.

While the invention has been described in connecting with preferred embodiments, it will be understood by those of ordinary skill in the art that other variations and modifications of the preferred embodiments described above may be made without departing from the scope of the invention. Other embodiments will be apparent to those of ordinary skill in the art from a consideration of the specification or practice of the invention disclosed herein. It is intended that the specification and the described examples are considered as exemplary only, with the true scope and spirit of the invention indicated by the following claims

1. A process for causing a myopic shift in the vision of a patient's eye, wherein said patient's eye comprises a cornea and said process comprises the step of:
instilling a bio-compatible material comprising a plurality of bio-compatible molecules to said cornea of said eye, wherein said cornea has a first composite refractive index, and said bio-compatible molecules alter said first composite refractive index to a second composite refractive index.

2. The process of claim 1, wherein said cornea has a first optical power and said bio-compatible molecules alter said first optical power to a second optical power.

3. The process of claim 1, wherein said bio-compatible molecules reduces a near point of accommodation of said cornea for a predetermined length of time.

4. The process of claim 3, wherein said predetermined length of time is less than or at least equal to about ten (10) minutes.

5. The process of claim 3, wherein said predetermined length of time is between about ten (10) minutes and about one (1) hour.

6. The process of claim 1, wherein said bio-compatible material is a hydrophilic polyanionic disaccharide.

7. The process of claim 6, wherein said hydrophilic polyanionic disaccharide is a sodium hyaluronate gel.

8. The process of claim 7, wherein said sodium hyaluronate gel is about a 1% sodium hyaluronate gel.

9. The process of claim 8, wherein an amount of said 1% sodium hyaluronate gel instilled to said cornea is in a range of about 0.05 ml to about 0.1 ml

10. The process of claim 1, wherein said bio-compatible molecules reduces a near point of accommodation of said cornea.

11. The process of claim 10, wherein said near point of accommodation is reduced for less than or at least equal to about ten (10) minutes.

12. The process of claim 10, wherein said near point of accommodation is reduced for between about ten (10) minutes and about one (1) hour.

13. The process of claim 10, wherein said first composite refractive index is about 1.3760.

14. The process of claim 10, wherein said cornea comprises a tear film and at least a portion of said bio-compatible molecules permeate said tear film.

15. The process of claim 14, wherein said patient's eye further comprises an inferior conjunctival sac and at least a portion of said bio-compatible material is instilled on said inferior conjunctival sac.

16. The process of claim 14, wherein said tear film comprises a tear mucin layer, wherein said tear mucin layer binds said bio-compatible molecules to said cornea.

17. The process of claim 1, wherein said second composite refractive index is greater than said first composite refractive index.

18. The process of claim 2, wherein said second optical power is greater than or equal to said first optical power.

19. A process for causing a myopic shift in the vision of a patient's eye, wherein said patient's eye comprises a cornea and said process comprises the step of:
instilling a bio-compatible material comprising a plurality of bio-compatible molecules to said cornea of said eye, wherein said cornea has a first composite refractive index, and said bio-compatible molecules alter said first composite refractive index to a second composite refractive index for a predetermined length of time.

20. The process of claim 19, wherein said cornea has a first optical power and said bio-compatible molecules alter said first optical power to a second optical power.

21. The process of claim 19, wherein said bio-compatible molecules reduces a near point of accommodation of said cornea

22. The process of claim 19, wherein said predetermined length of time is less than or at least equal to about ten (10) minutes.

23. The process of claim 19, wherein said predetermined length of time is between about ten (10) minutes and about one (1) hour.

24. The process of claim 19, wherein said bio-compatible material is a hydrophilic polyanionic disaccharide.

25. The process of claim 24, wherein said hydrophilic polyanionic disaccharide is a sodium hyaluronate gel.

26. The process of claim 25, wherein said sodium hyaluronate gel is about a 1% sodium hyaluronate gel.

27. The process of claim 24, wherein an amount of said 1% sodium hyaluronate gel instilled to said cornea is in a range of about 0.05 ml to about 0.1 ml.

28. The process of claim 19, wherein said bio-compatible molecules reduces a near point of accommodation of said cornea.

29. The process of claim 28, wherein said near point of accommodation is reduced for less than or at least equal to about ten (10) minutes.

30. The process of claim 28, wherein said near point of accommodation is reduced for between about ten (10) minutes and about one (1) hour.

31. The process of claim 28, wherein said first composite refractive index is about 1.3760.

32. The process of claim 28, wherein said cornea comprises a tear film and at least a portion of said bio-compatible molecules permeate said tear film.

33. The process of claim 32, wherein said patient's eye further comprises an inferior conjunctival sac and at least a portion of said bio-compatible material is instilled on said inferior conjunctival sac.

34. The process of claim 32, wherein said tear film comprises a tear mucin layer, wherein said tear mucin layer binds said bio-compatible molecules to said cornea.

35. The process of claim 19, wherein said second composite refractive index is greater than said first composite refractive index.

36. The process of claim 20, wherein said second optical power is greater than or equal to said first optical power.
